(19) United States
(12) Patent Application Publication
(10) Pub. No.: US 2015/0209550 A1
(43) Pub. Date: Jul. 30, 2015

(54) CATHETER HAVING DISSOLVABLE PORTIONS AND RELATED METHODS

(71) Applicant: B. BRAUN MELSUNGEN AG, Melsungen (DE)

(72) Inventors: E-Jen Teh, Penang (MY); Hui Kuun Teoh, Penang (MY)

(21) Appl. No.: 14/605,859

(22) Filed: Jan. 26, 2015

Related U.S. Application Data
(60) Provisional application No. 61/932,390, filed on Jan. 28, 2014.

(51) Int. Cl. A61M 25/00; A61L 29/14

(52) U.S. Cl. CPC A61M 25/0074; A61M 25/001; A61L 29/148; A61M 2210/12

(57) ABSTRACT

A catheter has a distal tip having a primary opening formed as the distal opening formed along the axis of the catheter. One or more secondary openings are formed through a side wall of the catheter proximal of the distal opening. A dissolvable portion made up of a resorbable material is disposed within the one or more secondary openings and aligned with the outer surface of the catheter so that the outer surface of the catheter and outer surface of the dissolvable portion combine to present a substantially smooth outer surface. When the catheter distal tip is arranged in a patient's blood vessel, the resorbable material dissolves, so that the fluid delivered by the catheter can exit the catheter through the primary and secondary openings. In some embodiments the secondary openings can be contiguous with the primary opening.

### CROSS-REFERENCE TO RELATED APPLICATIONS

The application claims priority to U.S. Provisional Application No. 61/932,390, which was filed on Jan. 28, 2014, the entirety of which is hereby incorporated by reference.

### BACKGROUND

The present disclosure relates to the field of infusion devices and methods of using such devices, and more particularly to intravenous (IV) catheters and methods of using such catheters.

Several types of medical therapies involve inserting a catheter into a patient's blood vessel and delivering treatment fluids into the patient's vasculature through the catheter. Catheter access to the vessel is generally accomplished through venipuncture, in which a catheter is disposed coaxially over a needle, and moves with the needle as it penetrates and is advanced through the patient's tissue so that eventually the needle and catheter distal tip are disposed within the patient's blood vessel. The needle can then be removed while the catheter distal tip remains in place, providing a conduit for fluid delivery.

A variety of fluids can be delivered to the patient's vasculature through the catheter. For example, fluids infused through catheters range from simple saline solutions to various medicaments to total parenteral nutrition. Such catheters can also be used to withdraw blood from the patient and/or to monitor various parameters of the patient's vascular system.

In some clinical circumstances it is desired to inject large quantities of fluid rapidly through the catheter and into the patient. For example, in some diagnostic imaging procedures, a “power injector” pump is used to inject viscous contrast media at a high flow rate to establish a contrast bolus or plug of contrast media in the bloodstream of the patient in order to enhance image quality. Also, in rapid infusion therapies, pressurized fluids are delivered through a catheter so that such fluid is delivered at a higher flow rate than in traditional infusion therapy. In such circumstances, increased backpressure can be expected in the infusion equipment and catheter assembly.

The catheter is often tapered at its distal tip in order to better facilitate the venipuncture process. A non-tapered catheter has increased risk of being snagged on tissues, thus increasing discomfort for the patient, and possibly fully or partially blocking the catheter from accompanying the needle into the blood vessel, which could result in a failed venipuncture attempt. In embodiments in which the catheter lumen is also tapered at the distal tip, the pressurized fluid may be accelerated as it exits the catheter, which can be undesirable. Some infusion catheters have apertures formed through a side wall of the catheter at or near the distal tip. Such apertures increase the delivery area, and can reduce exit flow speed and backpressure in the system. However, such apertures increase the risks of the catheter becoming snagged on tissue during insertion.

### SUMMARY

Catheters having one or more apertures formed through a side wall of the respective catheter at or adjacent its distal tip are disclosed, which one or more apertures are configured to avoid snagging on patient tissues during the insertion process, such as during venipuncture.

In accordance with one embodiment, a catheter assembly is provided comprising a catheter tube attached to a catheter hub. The catheter tube can comprise an elongated body defining a lumen, a distal tip defining a distal opening communicating with the lumen; and a tapered portion at and adjacent the distal tip. The tapered portion can comprise a dissolvable part and a non-dissolvable part

In accordance with another embodiment, the present invention provides a catheter assembly with a tubular catheter, which may simply be referred to as a catheter. The catheter comprises an elongated body defining a lumen, a distal tip defining a distal opening communicating with the lumen; and a tapered portion at and adjacent the distal tip. The tapered portion can comprise a dissolvable part and a non-dissolvable part.

In some such embodiments, surfaces of the non-dissolvable part and dissolvable part can be aligned so that the tapered portion has a smooth surface spanning both the non-dissolvable part and the dissolvable part. In other such embodiments, the non-dissolvable part can define the distal tip and extends from the distal tip to the elongated body. A hole can be formed through the non-dissolvable part proximal of the distal tip, and the dissolvable part can fill the hole.

In another embodiment, the non-dissolvable part can define a plurality of fingers, and the dissolvable part can be disposed between the fingers. In some such embodiments the fingers can be biased to a non-tapered configuration. The dissolvable part can hold the fingers in a tapered configuration so as to form the tapered portion. In yet further embodiments, the dissolvable part can define an outer surface of the distal tip about an entire circumference of the distal tip so that at least a portion of the fingers are covered by the dissolvable part.

In yet another embodiment, a flow rate of fluid through the catheter is greater when the dissolvable part has dissolved.

In some embodiments, the fingers can be biased outwardly relative to the elongated body. In other embodiments, the fingers can be integral with the elongate body.

In still further embodiments, the distal opening can have a first diameter, and the lumen can have a second diameter greater than the first diameter. In yet additional embodiments, the non-dissolvable part can comprise a lattice structure, and the lattice structure can support the dissolvable part.

In accordance with another embodiment, a method of delivering medical fluids is provided. The method includes advancing a distal tip of a catheter into a patient blood vessel. The catheter can have an elongated body defining a lumen. The distal tip can have a non-dissolvable part defining a primary exit opening aligned with an axis of the elongated body and communicating with the lumen, and a secondary exit opening formed through a side wall of the catheter and communicating with the lumen. A dissolvable part of the distal tip can cover the secondary exit opening. The dissolvable part of the catheter distal tip can be allowed to dissolve while within the patient's body so that the secondary exit opening is uncovered. The medical fluid can be delivered through the catheter primary and secondary exit openings after the dissolvable part has dissolved.

In some such embodiments, the primary and secondary exit opening can present a fluid exit area greater than the primary exit opening taken alone.

In additional embodiments, the secondary exit opening can be spaced from the primary exit opening.

In further embodiments, the primary and secondary exit openings can be contiguous with one another.

In yet further embodiments, the non-dissolvable part can define a plurality of fingers, and the dissolvable part can be disposed between the fingers.

In still further embodiments, the fingers can be held deflected radially inwardly by the dissolvable part, and when the dissolvable part dissolves, the fingers can move radially outwardly to a natural, at-rest position.

Yet another aspect of the present disclosure is a method of manufacturing a catheter assembly. The method can comprise: forming a catheter tube and attaching the catheter tube to a catheter hub; framing a tapered portion at a distal end of the catheter tube and terminating the distal end with a primary opening; forming an inchoate secondary opening with a dissolvable part adjacent the primary opening; and projecting a needle with a needle tip through the catheter tube so that a needle tip extends distal of the distal end of the catheter tube.

The method wherein the primary opening and the secondary opening when the dissolvable part dissolves can present a combined fluid exit area greater than the primary opening taken alone.

The method wherein the secondary opening can be spaced from the primary opening.

The method wherein the primary and secondary openings can be contiguous with one another.

The method wherein the primary opening can form with a non-dissolvable part and wherein the non-dissolvable part can define a plurality of fingers, and the dissolvable part can be disposed between the fingers.

The method wherein the fingers can be held deflected radially inwardly by the dissolvable part, and when the dissolvable part dissolves, the fingers can move radially outwardly to a natural, at-rest position.

### BRIEF DESCRIPTION OF THE DRAWINGS

FIG. 1 is a perspective view of one embodiment of a catheter tip wherein a hole is formed through a side wall thereof;

FIG. 2 is a perspective view of the catheter tip of FIG. 1 in which the hole is filled with a resorbable material;

FIG. 3 is a perspective view of a catheter tip configured in accordance with another embodiment and comprising a resorbable material;

FIG. 4 is a side view of the catheter tip of FIG. 3, shown after the resorbable material has dissolved;

FIG. 5 is a perspective view of a catheter tip configured in accordance with yet another embodiment and comprising a resorbable material;

FIG. 6 is a side view of the catheter tip of FIG. 5, shown after the resorbable material has dissolved;

FIG. 7 is a side view of a catheter tip configured in accordance with still another embodiment and comprising a resorbable material;

FIG. 8 is a side view of the catheter tip of FIG. 7, shown after the resorbable material has dissolved; and

FIG. 9 is a side view of a catheter distal tip configured in accordance with a still further embodiment.

### DETAILED DESCRIPTION

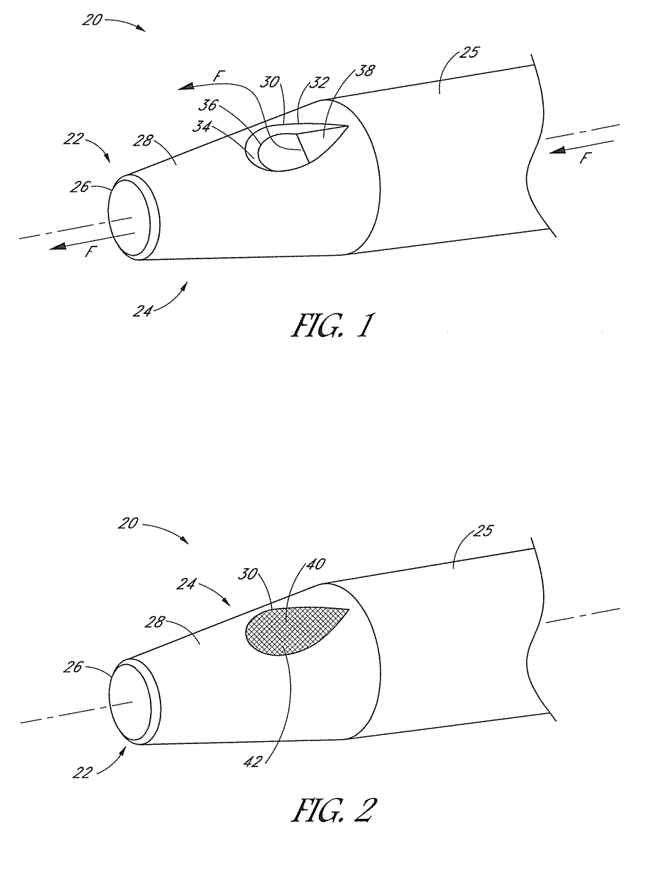

With initial referenced to FIG. 1, a tubular catheter 20, herein catheter tube or simply catheter, can be configured for use diffusing a fluid such as, for example, radioactive contrast during a diagnostic or other procedure. It is desired to deliver the fluid at a desired flow rate. Thus, a catheter 20 is chosen having a sufficient lumen diameter to deliver fluid at the desired flow rate. In the illustrated embodiment, a distal tip 22 of the catheter 20 includes a tapered portion 24 distal of a catheter body 25. A distal lumen opening 26 is defined at the distal end of the distal tip 22 and aligned with an axis of the catheter. In some embodiments, the catheter lumen is tapered in the distal tip 22 adjacent the distal lumen opening 26. In other embodiments, the catheter lumen diameter remains constant through the distal tip 22. Preferably, the catheter is made of a flexible, medical grade polymer. Although not shown, the catheter 20 is understood to be attached to a catheter hub. In a ready position before placement of the catheter 20 into a patient's vein, the catheter 20 is part of a catheter assembly, which comprises a catheter hub having the catheter tube 20 attached therewith, a needle with a sharp needle tip projecting through the catheter tube, and wherein the needle is connected to a needle hub. In some embodiments, the catheter hub may be equipped with a septum or a valve to limit or restrict fluid flow across the catheter hub. In other examples, a needle guard is provided to cover the needle tip. The needle guard may be a retractable type that retracts the needle and needle tip into a protective housing, with or without a spring, or a clip type mounted on the needle and slidable on the needle shaft to cover the needle tip. For the clip type, a change in profile, such as a crimp or a bulge, may be incorporated proximal of the needle tip for engaging a perimeter defining an opening on the needle guard. Exemplary catheter assemblies are shown in U.S. Pat. No. 8,333,735, the contents of which are expressly incorporated herein by reference.

In the embodiment shown in FIG. 1, one or more secondary openings 30 are formed through the catheter side wall proximal of the distal opening 26. This increases the exit flow area, and thus helps to reduce system backpressure and fluid exit speed, even when the catheter's fluid flow rate is increased. However, during venipuncture, surrounding tissue may bulge into the secondary openings 30. This may result in snags or tissue shears as the catheter 20 is pushed past such tissues. The snags and tissue shears may cause the catheter 20 to not properly enter into the blood vessel and/or may cause increased pain to the patient.

In FIG. 1, the secondary opening 30 is configured to minimize the likelihood of snags and shears. For example, the secondary opening 30 includes a tear-shaped opening 32 on the surface 28 of the catheter 20. As shown, the surface 28 may be referred to as the distal cone surface of the catheter. A leading surface 34 depends from the tear-shaped opening 32 and leads to a D-shaped hole 36 that is spaced from the surface 28 of the catheter 20. On the proximal end of the D-shaped hole 36, an inclined proximal surface 38 is angled relatively gently from the D-shaped hole 36 to the catheter surface 28. As such, in the illustrated embodiment, hole 36 is smaller in diameter than the opening 32 formed at the surface of the catheter. This structure attempts to prevent tissues that may bulge into the secondary opening 30 from actually entering the D-shaped hole 36, and further gently urges such bulging tissues out of the secondary opening 30 via the inclined proximal surface 38. Such structure attempts to avoid snags and shears. However, tissues can sometimes be unpredictable, and snags and shears may still occur notwithstanding use of structures intended to direct such tissues away from holes through the catheter side wall.

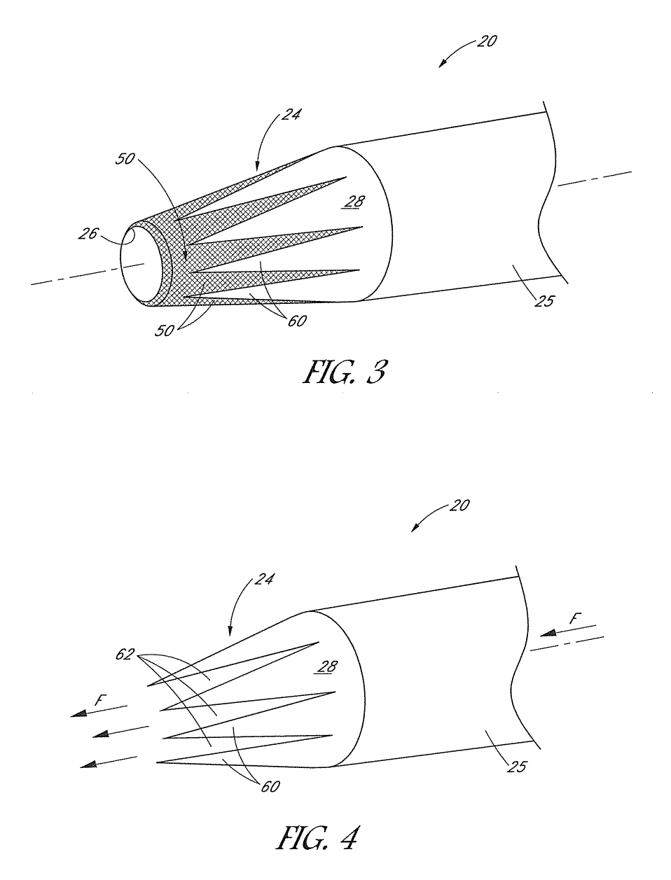

With reference next to FIG. 2, the catheter 20 of FIG. 1 is shown, except that the secondary opening 30 is filled with a plug 40 made of a bioresorbable material such as any of many bioresorbable polymers currently available for medical use. In the illustrated embodiment, the plug 40 is shaped so that the surface 42 of the plug 40 is smooth and aligned with the catheter surface 28 so that surfaces 28 and 42 present a continuous surface or substantially continuous. As such, during venipuncture, the patient's tissues will slide over the catheter and plug surfaces 28, 42 with substantially no more risk of snags or shears than with a typical catheter having no secondary openings.

Preferably the plug 40 is sufficiently thick so as to maintain strength to withstand the venipuncture without failing or degrading, but sufficiently thin so that the plug 40 dissolves relatively quickly. For example, it is desired that the plug 40 dissolve in a very short time, such as within a few minutes of the catheter tip 22 entering the bloodstream. Different compounds and materials may be used in various thicknesses and percent to vary the dissolve time. Preferably the rest of the catheter is made of a material that is not substantially resorbable. Once the plug 40 has dissolved, the secondary opening 30 is available (along with the distal opening 26) for delivery of fluids F, such as contrast or other therapeutic fluids, and the fluid delivery flow rate potential of the catheter 20 can be substantially increased. More specifically, provision of secondary openings reduces fluid exit speed for a particular fluid flow delivery rate when compared to fluid exit speeds for an equivalent catheter having only the traditional distal opening 26. Thus, an aspect of the present disclosure is understood to include a catheter assembly comprising a catheter tube attached to a catheter hub and wherein the catheter tube comprises a distal opening and a side opening to permit outflows or inflows through the catheter tube from at least two different openings. The distal opening may be referred to as a first opening and the side opening may be referred to as a second opening. As disclosed in this embodiment, the side opening is an inchoate opening that forms a complete opening adequate for fluid flow following a short period to permit a plug to dissolve to form the second opening. Additional side inchoate openings, such as second or third side openings, may be incorporated each with a plug that is dissolvable.

In the illustrated embodiment the secondary opening 30 is formed in the tapered portion 24 of the catheter 20 adjacent the distal tip 22. It is to be understood that other embodiments may employ multiple secondary openings in the tapered portion 24, in the non-tapered body portion 25 proximal of the tapered portion 24, or both.

In FIG. 2, the plug 40 is placed in a secondary opening 30 having the shape and structure of FIG. 1. It is to be understood that, in other embodiments, the secondary opening may embody a hole with no depending or inclined surfaces, and having various shapes such as circular, oval, irregular, teardrop or the like.

In the illustrated embodiment, surfaces such as the leading surface 34 and the inclined proximal surface 38 can provide support for the resorbable plug 40, helping maintain the plug 40 in place during formation of the plug, insertion of the catheter during venipuncture, and for a portion of the time it takes for the resorbable material to dissolve. It is to be understood that other aperture structures can be provided to help support the plug. For example, in still other embodiments the secondary opening can include a lattice or mesh structure that helps to support the plug 40, but still enables fluid flow once the plug has dissolved. The plug may be formed by placing a stem inside the lumen of the catheter tube and coating or spraying the secondary opening 30 with a biocompatible material that dissolves when come in contact with body fluids.

Resorbable materials that are believed to be usable to implement the present catheter tubes with both a dissolvable part and a non-dissolvable part include, without limitation, Polylactides (PLA), Polyglycolides (PGA), Polycaprolactones (PCL), Polydioxanone (PDS), poly(lactide-co-glycolide) (PLGA), Poly vinyl pyrrolidone (PVP), Trimethylcarbonate (TMC), and Copolymer blends of these materials. Poly(esters) based on polylactide (PLA), polyglycolide (PGA), polycaprolactone (PCL), and their copolymers are known to have been employed as biomaterials. Degradation of these materials yields the corresponding hydroxy acids, making them safe for in vivo use. Other bio- and environmentally degradable polymers include poly(hydroxyalkanoate)s of the PHB-PHV class, additional poly(ester)s, and natural polymers, e.g., hyaluronan and particularly, modified poly(saccharide)s, e.g., starch, cellulose, and chitosan.

For other catheter assemblies and assembly components disclosed herein below, such as for other catheter tubes, it is understood that where a feature is shown but not expressly described and is otherwise the same or similar to the feature or features described elsewhere, such as above with reference to FIGS. 1 and 2, the disclosed part or parts shown in the subsequent drawing figures but not expressly described because of redundancy and because knowledge is built on a foundation laid by earlier disclosures may nonetheless be understood to be described or taught by the same or similar features expressly set forth in the text for the embodiments in which the feature or features are described, such as for the catheter assemblies and catheter components of FIGS. 1 and 2. Said differently, subsequent disclosures of the present application are built upon the foundation of earlier disclosures unless the context indicates otherwise. The disclosure is therefore understood to teach a person of ordinary skill in the art the disclosed embodiments and the features of the disclosed embodiments without having to repeat similar components and features in all embodiments since a skilled artisan would not disregard similar structural features having just read about them in several preceding paragraphs nor ignore knowledge gained from earlier descriptions set forth in the same specification. As such, the same or similar features shown in the following catheter assemblies incorporate the teachings of earlier embodiments unless the context indicates otherwise. Therefore, it is contemplated that later disclosed embodiments enjoy the benefit of earlier expressly described embodiments, such as features and structures of earlier described embodiments, unless the context indicates otherwise.

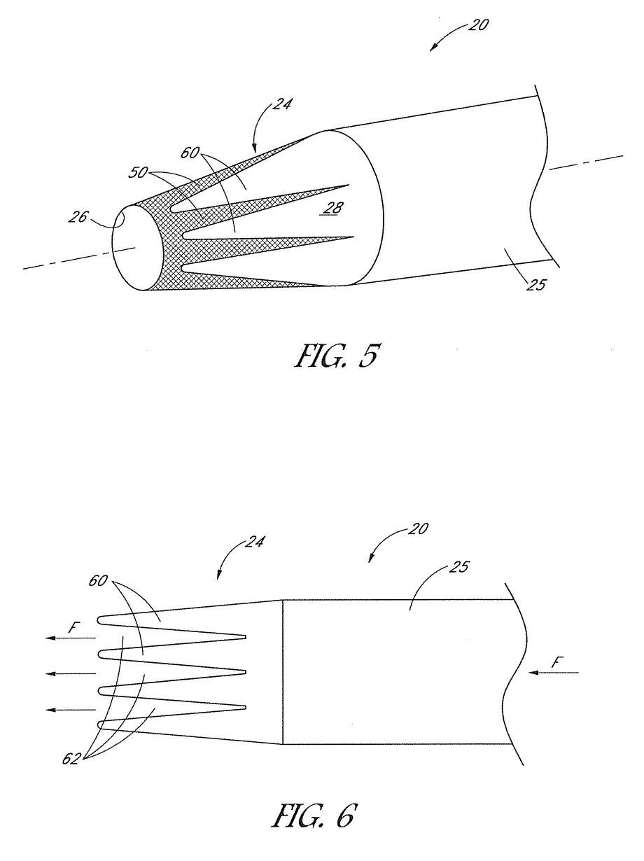

With reference next to FIGS. 3 and 4, in another embodiment, a main body 25 of a catheter 20 is made of a first flexible material such as a flexible plastic material which can include, for example, polyethylene tubing. A plurality of fingers 60 are formed at a distal end of the plastic tube 25. The fingers 60 are formed into a desired distally-tapered configuration, but with spaces 62 defined between the fingers. A dissolvable portion 50 of the tapered distal portion 24 comprises resorbable material arranged between and aligned with the fingers 60 to form the tapered portion 24. As such, the resulting catheter 20 has a tapered portion 24 at and adjacent the distal tip 22 having a smooth surface that will readily perform like a typical catheter during venipuncture, and venipuncture proceeds with little or no increased risk of snags and shears relative to a typical tapered-tip catheter 20.

With specific reference to FIG. 4, once the distal tapered portion 24 of the catheter 20 enters the blood stream, the dissolvable portion 50 melts away, strips away, washes away, or simply disappears leaving the fingers 60. As such, fluid F can now exit the catheter through the spaces 62 between the fingers 60 in addition to the distal opening 26. In such embodiments, the spaces 62 can be considered secondary openings through the side wall of the catheter, being secondary to the distal opening 26, which can be considered a primary opening. Thus, in such embodiments, the primary and secondary openings are contiguous, but the secondary openings are closed by the dissolvable portion 50 prior to placing the catheter into the patient's bloodstream.

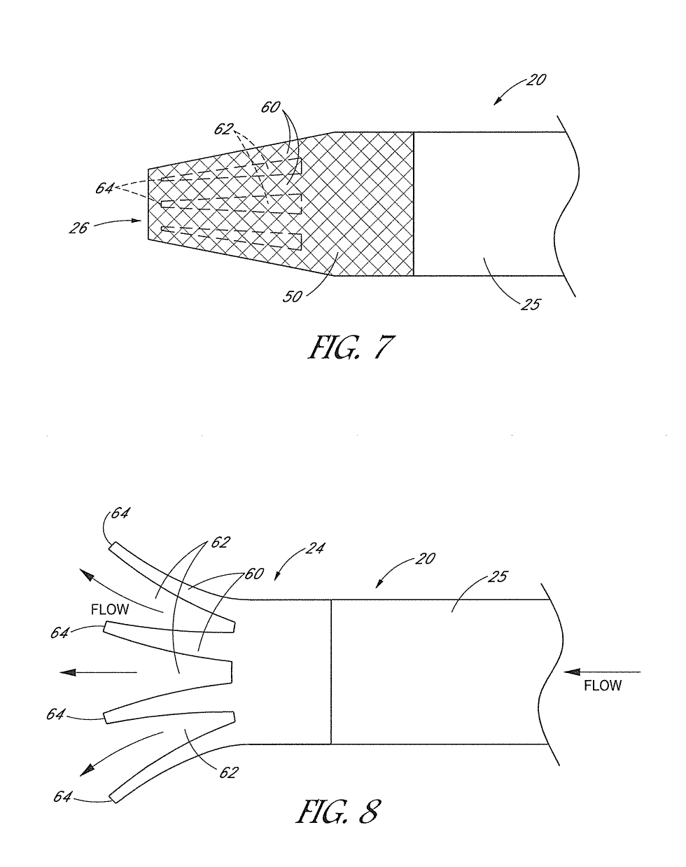

With reference next to FIGS. 5 and 6, another embodiment is illustrated in which the fingers 60 are biased toward an “open” condition (see FIG. 6) in which the fingers 60 are directed in substantially the same direction as the body 25 of the catheter 20. Such a catheter can be made, for example, by simply cutting the fingers 26 in an existing, uniform-diameter catheter. During manufacture the fingers 60 are urged inwardly to form a tapered distal tip 24, and resorbable materials added both to form the dissolvable portion 50 and to hold the fingers 60 in the tapered disposition so that the fingers 60 and dissolvable portion 50 together form the tapered distal tip 24. Once the catheter is placed within the patient's vessel, the dissolvable portion 50 melts away, releasing the fingers 60 so that they are free to expand to their natural, at-rest position as shown in FIG. 6. As such, the catheter tube's output opening is substantially the same as the catheter lumen diameter in the body 25, and the catheter distal tip 22 has no restraining effect on fluid flow rate through the catheter 20.

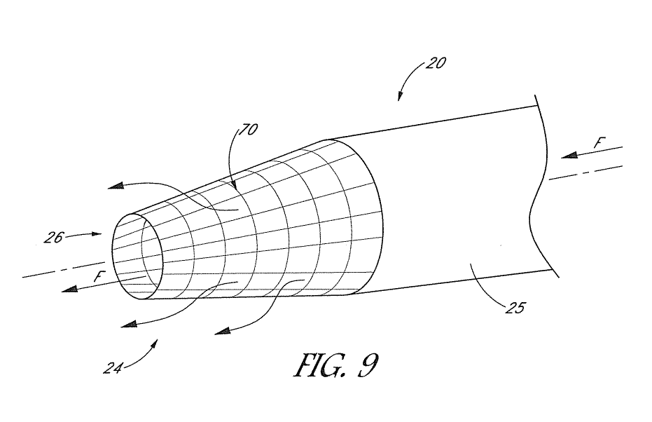

With reference next to FIGS. 7-8, in still other embodiments, the catheter fingers 60 may be biased radially outwardly from the catheter body 25 (see FIG. 8), and in some embodiments can be splayed relative to the catheter body 25. In some such embodiments, tips 64 of the fingers 60 may be blunt so as to minimize the likelihood of damaging a vessel wall should they contact the vessel wall. In manufacture, the fingers 60 are deflected radially inwardly to form the tapered distal portion, and resorbable material making up the dissolvable portion can partially or completely cover the fingers 60 (see FIG. 7) so as to maintain the fingers 60 in a deflected position and to define the tapered distal tip 24.

In the illustrated embodiment the distal portion 50 defines the entire outer surface of the tapered distal tip 24, and the fingers 60 are substantially or completely covered by the dissolvable portion 50. Of course, parts of the dissolvable portion can extend into and through the spaces 62 between the fingers 60. After the catheter 20 is placed in the patient's blood vessel, the dissolvable portion will dissolve, freeing the fingers to return to their natural position, which in the illustrated embodiment is splayed outwardly relative to the catheter body 25.

With reference next to FIG. 9, in another embodiment, a catheter 20 may include a non-dissolvable lattice structure 70 which forms all or part of the tapered distal tip portion 24. Dissolvable material 50 (not shown) can be deposited onto the lattice structure 70, and will be supported by the lattice structure 70, to form the tapered distal tip 24. Preferably, the lattice structure 70 is shaped as a mesh representing the desired distal tip taper. During manufacture, the lattice structure 70 can be inserted into a mold, and resorbable material can be added so form the dissolvable portion, which dissolvable portion will define the smooth, tapered outer surface of the distal tip 24, thus facilitating uneventful venipuncture. The lattice structure 70 provides support, strength, and shape for the dissolvable material 50 that defines the distal tip surface.

Once the catheter is positioned in the blood vessel, the dissolvable portion will dissolve, leaving the catheter 20 with the lattice structure 70 as depicted in FIG. 9. A mesh-based lattice structure has several openings 62 defined between elements of the mesh. In fact, fluid flow F can be expected to proceed as though the exit opening is the same as the lumen diameter throughout the catheter body 25.

The embodiments discussed above have disclosed structures with substantial specificity. This has provided a good context for disclosing and discussing inventive subject matter. However, it is to be understood that other embodiments may employ different specific structural shapes and interactions. Also, although embodiments discussed herein have been disclosed as appropriate for high pressure and flow rates (i.e., over 1 L/hr) associated with rapid infusion, it is to be understood that the principles discussed herein can be employed with catheter assemblies intended for more traditional, relatively low infusion flow rates (i.e., under 1 L/hr). Further, embodiments employing a resorbable material can be used at high or low infusion flow rates, or even when there is very little or no fluid flow or back pressure.

In some preferred embodiments, during use of any of the above-described structural embodiments and variations thereof, after venipuncture, the clinician will wait a period of time before beginning infusion in order to provide time for the resorbable material to at least partially (and preferably substantially completely) dissolve. Depending on the resorbable material, such wait times can be from a few seconds to a few or several minutes. In some other embodiments, infusion at low flow rates can begin while the resorbable material is dissolving, and flow rates can be increased over time as dissolving becomes more complete.

Methods of making and of using the various catheter assemblies and there components are understood to be within the scope of the present disclosure.

Although inventive subject matter has been disclosed in the context of certain preferred or illustrated embodiments and examples, it will be understood by those skilled in the art that the inventive subject matter extends beyond the specifically disclosed embodiments to other alternative embodiments and/or uses of the invention and obvious modifications and equivalents thereof In addition, while a number of variations of the disclosed embodiments have been shown and described in detail, other modifications, which are within the scope of the inventive subject matter, will be readily apparent to those of skill in the art based upon this disclosure. For example, the principles discussed herein may be practiced in catheters used in clinical treatments outside the patient's bloodstream. It is also contemplated that various combinations or subcombinations of the specific features and aspects of the disclosed embodiments may be made and still fall within the scope of the inventive subject matter. For example, in some embodiments having features resembling the embodiments illustrated in FIG. 3 or 5, the distal tip can be partially or entirely coated by the resorbable material as in the embodiment illustrated in FIG. 7. Accordingly, it should be understood that various features and aspects of the disclosed embodiments can be combined with or substituted for one another in order to form varying modes of the disclosed inventive subject matter. Thus, it is intended that the scope of the inventive subject matter herein disclosed should not be limited by the particular disclosed embodiments described above, but should be determined only by a fair reading of the claims that follow.

What is claimed is:

1. A catheter assembly, comprising:
a catheter tube comprising an elongated body defining a lumen;
a distal tip defining a distal opening communicating with the lumen; and
a tapered portion at and adjacent the distal tip;
wherein the tapered portion comprises a dissolvable part and a non-dissolvable part.

2. A catheter assembly as in claim 1, wherein surfaces of the non-dissolvable part and dissolvable part are aligned so that the tapered portion has a smooth surface spanning both the non-dissolvable part and the dissolvable part.

3. A catheter assembly as in claim 1, wherein the non-dissolvable part defines the distal tip and extends from the distal tip to the elongated body, a hole being formed through the non-dissolvable part proximal of the distal tip, and the dissolvable part fills the hole.

4. A catheter assembly as in claim 1, wherein the non-dissolvable part defines a plurality of fingers, and the dissolvable part is disposed between the fingers.

5. A catheter assembly as in claim 4, wherein the fingers are biased to a non-tapered configuration, and the dissolvable part holds the fingers in a tapered configuration so as to form the tapered portion.

6. A catheter assembly as in claim 5, wherein the dissolvable part defines an outer surface of the distal tip about an entire circumference of the distal tip so that at least a portion of the fingers are covered by the dissolvable part.

7. A catheter assembly as in claim 5, wherein a flow rate of fluid through the catheter is greater when the dissolvable part has dissolved.

8. A catheter assembly as in claim 5, wherein the fingers are biased outwardly relative to the elongated body.

9. A catheter assembly as in claim 5, wherein the fingers are integral with the elongated body.

10. A catheter assembly as in claim 1, wherein the distal opening has a first diameter, and the lumen has a second diameter greater than the first diameter.

11. A catheter assembly as in claim 10, wherein the non-dissolvable part comprises a lattice structure, and the lattice structure supports the dissolvable part.

12. A method of manufacturing a catheter assembly, comprising:
forming a catheter tube and attaching the catheter tube to a catheter hub;
forming a tapered portion at a distal end of the catheter tube and terminating the distal end with a primary opening;
forming an inchoate secondary opening with a dissolvable part adjacent the primary opening; and
projecting a needle with a needle tip through the catheter tube so that a needle tip extends distal of the distal end of the catheter tube.

13. A method as in claim 12, wherein the primary opening and the secondary opening when the dissolvable part dissolves present a combined fluid exit area greater than the primary opening taken alone.

14. A method as in claim 13, wherein the secondary opening is spaced from the primary opening.

15. A method as in claim 13, wherein the primary and secondary openings are contiguous with one another.

16. A method as in claim 13, wherein the primary opening is formed with a non-dissolvable part and wherein the non-dissolvable part defines a plurality of fingers, and the dissolvable part is disposed between the fingers.

17. A method as in claim 16, wherein the fingers are held deflected radially inwardly by the dissolvable part, and when the dissolvable part dissolves, the fingers move radially outwardly to a natural, at-rest position.
